(19) United States
(12) Patent Application Publication
(10) Pub. No.: US 2025/0377375 A1
(43) Pub. Date: Dec. 11, 2025

(54) ULTRASOUND PROBE ACTIVATING METHOD AND APPARATUS, ULTRASOUND IMAGING DEVICE, AND MICRO CONTROLLER UNIT

(71) Applicant: WUHAN UNITED IMAGING HEALTHCARE CO., LTD., Wuhan, Hubei (CN)

(72) Inventors: Di Huang, Wuhan (CN); Lu Cai, Wuhan (CN)

(21) Appl. No.: 19/230,132

(22) Filed: Jun. 6, 2025

(30) Foreign Application Priority Data
Jun. 11, 2024 (CN) 202410749777.1

(51) Int. Cl. G01P 1/02; G01N 29/24; G05B 19/042; G06F 3/01

(52) U.S. Cl. CPC G01P 1/023; G01N 29/24; G05B 19/0423; G06F 3/011

(57) ABSTRACT

An ultrasound probe activating method, an apparatus, an ultrasound imaging device, and a micro controller unit are provided. The method includes: acquiring a real-time acceleration of the ultrasound probe detected by the motion sensor, in a case that the real-time acceleration meets a preset condition, sending a probe activating instruction to the ultrasound host, so that the ultrasound host activates the ultrasound probe. The real-time acceleration includes a real-time linear acceleration and a real-time rotational acceleration. The preset condition includes that the real-time linear acceleration is not matched with an initial linear acceleration of the ultrasound probe, and the real-time rotational acceleration exceeds a first preset threshold.

### CROSS-REFERENCE TO RELATED APPLICATION

This application claims priority to Chinese patent applications No. 202410749777.1, filed on Jun. 11, 2024, titled “ULTRASOUND PROBE ACTIVATING METHOD AND APPARATUS, ULTRASOUND IMAGING DEVICE, AND MICRO CONTROLLER UNIT”, the content of which is hereby incorporated herein in its entirety by reference.

### TECHNICAL FIELD

The present disclosure generally relates to the field of medical devices, and in particular, to an ultrasound probe activating method, an apparatus, an ultrasound imaging device, and a micro controller unit.

### BACKGROUND

Medical ultrasound imaging devices with full functions (e.g., full-body ultrasound machines, gynecologic ultrasound machines, etc.) are widely used in clinical applications since the devices can provide more comprehensive examination. The medical ultrasound imaging devices with full functions support the use of multiple ultrasound probes, but usually only one ultrasound probe is activated at a scanning moment, while other ultrasound probes are off without applying an excitation signal.

In the related art, when a doctor who performs a scanning operation needs to switch an ultrasound probe, an unused ultrasound probe is usually placed back on a shelf, a to-be-used ultrasound probe is picked up, and an activating switch is triggered by a functional button or a touchscreen on an operation panel of an ultrasound host, so as to activate the ultrasound probe. This operation is relatively cumbersome, reducing convenience of using the ultrasound imaging devices.

Therefore, in a current ultrasound probe activating technology, operations are cumbersome and the ultrasound probes are not easy to use.

### SUMMARY

According to various embodiments of the present disclosure, an ultrasound probe activating method, an apparatus, an ultrasound imaging device, a micro controller unit, a computer-readable storage medium, and a computer program product are provided.

In a first aspect, an ultrasound probe activating method is provided in the present disclosure. The method includes: acquiring a real-time acceleration of an ultrasound probe detected by a motion sensor, in a case that the real-time acceleration meets a preset condition, sending a probe activating instruction to an ultrasound host, so that the ultrasound host activates the ultrasound probe. The motion sensor is disposed on the ultrasound probe. The real-time acceleration includes a real-time linear acceleration and a real-time rotational acceleration. The preset condition includes that the real-time linear acceleration is not matched with an initial linear acceleration of the ultrasound probe, and the real-time rotational acceleration exceeds a first preset threshold.

In an embodiment, the real-time linear acceleration includes three real-time linear components and the real-time rotational acceleration includes three real-time rotational components. The preset condition further includes that any one of the three real-time linear components of the real-time linear acceleration is not matched with a corresponding initial linear component, and any one of the three real-time rotational components of the real-time rotational acceleration exceeds the first preset threshold.

In an embodiment, in the case that the real-time acceleration meets the preset condition, sending the probe activating instruction to the ultrasound host further includes: when a difference between any one of the three real-time linear components of the real-time linear acceleration and a corresponding initial linear component exceeds a second preset threshold, comparing each of the three real-time rotational components of the real-time rotational acceleration with the first preset threshold; when any one of the three real-time rotational components of the real-time rotational acceleration exceeds the first preset threshold, sending the probe activating instruction to the ultrasound host.

In an embodiment, in the case that the real-time acceleration meets the preset condition, sending the probe activating instruction to the ultrasound host further includes: in the case that the real-time acceleration meets the preset condition, updating an original displacement count value to obtain an updated displacement count value, sending the updated displacement count value to the ultrasound host, and activating, by the ultrasound host, the ultrasound probe according to the updated displacement count value.

In an embodiment, updating the original displacement count value includes increasing the original displacement count value by a preset value.

In an embodiment, before acquiring the real-time acceleration of the ultrasound probe detected by the motion sensor, the method further includes: reading an initial acceleration of the ultrasound probe from a register of the motion sensor in response to a received interrupt instruction, and extracting the initial linear acceleration of the ultrasound probe from the initial acceleration. The interrupt instruction is sent by the motion sensor when the motion sensor perceives a gravity acceleration of the ultrasound probe.

In an embodiment, acquiring the real-time acceleration of the ultrasound probe detected by the motion sensor further includes: reading a register of the motion sensor in real time to obtain the real-time acceleration of the ultrasound probe.

In a second aspect, an ultrasound probe activating apparatus is further provided in the present disclosure. The apparatus includes: means for acquiring a real-time acceleration of an ultrasound probe detected by a motion sensor, and means for sending a probe activating instruction to an ultrasound host in a case that the real-time acceleration meets a preset condition, so that the ultrasound host activates the ultrasound probe. The motion sensor is disposed on the ultrasound probe. The real-time acceleration includes a real-time linear acceleration and a real-time rotational acceleration. The preset condition includes that the real-time linear acceleration is not matched with an initial linear acceleration of the ultrasound probe, and the real-time rotational acceleration exceeds a first preset threshold.

In a third aspect, an ultrasound imaging device is further provided in the present disclosure, including an ultrasound host and an ultrasound probe. A micro controller unit and a motion sensor are disposed on the ultrasound probe, the micro controller unit is connected to the motion sensor and the ultrasound host, respectively. The motion sensor is configured for perceiving a real-time acceleration of the ultrasound probe and sending information of the real-time acceleration to the micro controller unit, and the real-time acceleration includes a real-time linear acceleration and a real-time rotational acceleration. The micro controller unit is configured for sending a probe activating instruction to the ultrasound host in a case that the real-time acceleration meets a preset condition. The preset condition includes that the real-time linear acceleration is not matched with an initial linear acceleration of the ultrasound probe, and the real-time rotational acceleration exceeds a first preset threshold. The ultrasound host is configured for activating the ultrasound probe when receiving the probe activating instruction.

In a fourth aspect, a micro controller unit is further provided in the present disclosure, including a memory and a processor. A computer program is stored in the memory, and the processor is configured to execute the computer program to implement the following step: acquiring a real-time acceleration of an ultrasound probe detected by a motion sensor, in a case that the real-time acceleration meets a preset condition, sending a probe activating instruction to an ultrasound host, so that the ultrasound host activates the ultrasound probe. The motion sensor is disposed on the ultrasound probe. The real-time acceleration includes a real-time linear acceleration and a real-time rotational acceleration. The preset condition includes that the real-time linear acceleration is not matched with an initial linear acceleration of the ultrasound probe, and the real-time rotational acceleration exceeds a first preset threshold.

In a fifth aspect, a computer-readable storage medium is further provided in the present disclosure, which stores a computer program. The computer program is executed by a processor to implement the following steps: acquiring a real-time acceleration of an ultrasound probe detected by a motion sensor, in a case that the real-time acceleration meets a preset condition, sending a probe activating instruction to an ultrasound host, so that the ultrasound host activates the ultrasound probe. The motion sensor is disposed on the ultrasound probe. The real-time acceleration includes a real-time linear acceleration and a real-time rotational acceleration. The preset condition includes that the real-time linear acceleration is not matched with an initial linear acceleration of the ultrasound probe, and the real-time rotational acceleration exceeds a first preset threshold.

In a sixth aspect, a computer program product is further provided in the present disclosure, which includes a computer program. The computer program is executed by a processor to implement the following steps: acquiring a real-time acceleration of an ultrasound probe detected by a motion sensor, in a case that the real-time acceleration meets a preset condition, sending a probe activating instruction to an ultrasound host, so that the ultrasound host activates the ultrasound probe. The motion sensor is disposed on the ultrasound probe. The real-time acceleration includes a real-time linear acceleration and a real-time rotational acceleration. The preset condition includes that the real-time linear acceleration is not matched with an initial linear acceleration of the ultrasound probe, and the real-time rotational acceleration exceeds a first preset threshold.

In the above ultrasound probe activating method, the apparatus, the ultrasound imaging device, the micro controller unit, the computer-readable storage medium, and the computer program product, the real-time acceleration of the ultrasound probe detected by the motion sensor is acquired, in the case that the real-time acceleration meets the preset condition, the probe activating instruction is sent to the ultrasound host, so that the ultrasound host activates the ultrasound probe. The real-time acceleration includes the real-time linear acceleration and the real-time rotational acceleration. The preset condition includes that the real-time linear acceleration is not matched with the initial linear acceleration of the ultrasound probe, and the real-time rotational acceleration exceeds the first preset threshold. The real-time acceleration of the ultrasound probe is used to trigger the ultrasound host to activate the ultrasound probe when the ultrasound probe is picked up. In other words, when the real-time linear acceleration of the ultrasound probe changes and the real-time rotational acceleration exceeds the first preset threshold, the ultrasound host is automatically controlled to activate the ultrasound probe, so that no manual activation of the ultrasound probe is required, thereby increasing convenience of using the ultrasound imaging device.

### BRIEF DESCRIPTION OF THE DRAWINGS

In order to more clearly illustrate the technical solutions in the embodiments of the present disclosure or the related technology, the accompanying drawings to be used in the description of the embodiments or the related technology will be briefly introduced below, and it will be obvious that the accompanying drawings in the following description are only some of the embodiments of the present disclosure, and that, for one skilled in the art, other accompanying drawings can be obtained based on these accompanying drawings without putting in creative labor.

FIG. 1 is a flowchart of an ultrasound probe activating method in an embodiment.

FIG. 2 is a schematic diagram of an ultrasound probe on which a motion sensor is mounted in an embodiment.

FIG. 3 is a schematic circuit diagram of an ultrasound imaging device in an embodiment.

FIG. 4 is a schematic diagram of a six-axis MEMS sensor in an embodiment.

FIG. 5 is a flowchart of an MCU firmware program in an embodiment.

FIG. 6 is a flowchart of a probe pickup detecting program in an embodiment.

FIG. 7 is a flowchart of an ultrasound probe activating method in another embodiment.

FIG. 8 is a schematic block diagram of an ultrasound probe activating apparatus in an embodiment.

FIG. 9 is a schematic block diagram of an ultrasound imaging device in an embodiment.

FIG. 10 is an internal structural diagram of a micro controller unit in an embodiment.

### DETAILED DESCRIPTION OF THE EMBODIMENT

To make objectives, technical solutions, and advantages of the present disclosure clearer, the following further describes the present disclosure in detail with reference to the accompanying drawings and embodiments. It should be understood that the specific embodiments described herein are merely used to explain the present disclosure, and are not intended to limit the present disclosure.

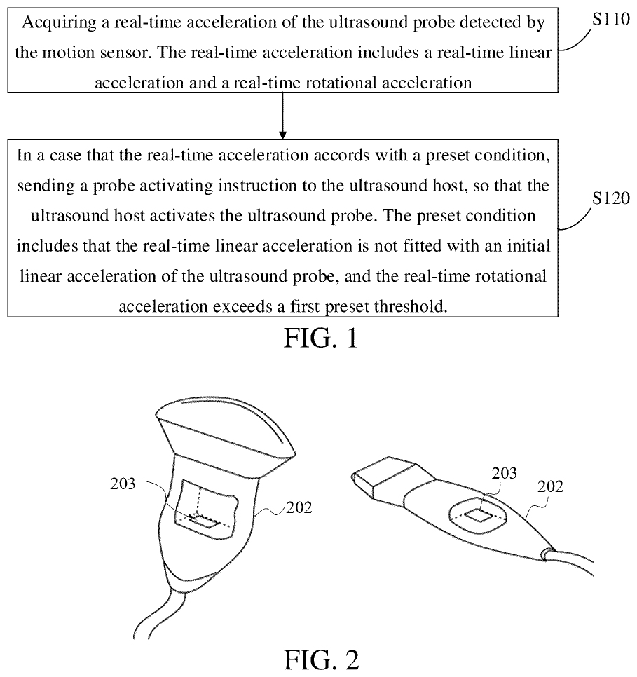

In an exemplary embodiment, referring to FIG. 1, an ultrasound probe activating method is provided. The method is applied to a micro controller unit. The micro controller unit is disposed on an ultrasound probe of an ultrasound imaging device, a motion sensor is further disposed on the ultrasound probe, and the micro controller unit is connected to the motion sensor and an ultrasound host of the ultrasound imaging device, respectively. In the present embodiment, the method includes the following step 110 and step 120.

Step 110 includes acquiring a real-time acceleration of the ultrasound probe detected by the motion sensor. The real-time acceleration includes a real-time linear acceleration and a real-time rotational acceleration.

The motion sensor may include a multi-axis sensor, including but not limited to a six-axis sensor or a nine-axis sensor, or may be specifically a MEMS (Micro-Electro-Mechanical System) sensor.

The real-time acceleration may include an acceleration detected in real time. The real-time linear acceleration may include a real-time detected linear acceleration. The real-time rotational acceleration may include a real-time detected rotational acceleration, and the rotational acceleration may include an angular acceleration.

Alternatively, the ultrasound image device may include an ultrasound host and an ultrasound probe, and the micro controller unit and the motion sensor are disposed on the ultrasound probe. The micro controller unit and the motion sensor may communicate with each other, and the micro controller unit and the ultrasound host may communicate with each other. Taking the six-axis sensor as an example, the motion sensor may transmit information of a six-axis acceleration that is detected in real time to the micro controller unit. The micro controller unit may take a received six-axis acceleration as a real-time acceleration of the ultrasound probe, take a three-axis linear acceleration in the six-axis acceleration as a real-time linear acceleration, and take a three-axis rotational acceleration in the six-axis acceleration as a real-time rotational acceleration.

FIG. 2 is a schematic diagram of an ultrasound probe on which a motion sensor is mounted. Referring to FIG. 2, a groove may be disposed on an ultrasound probe 202, and a carrier plate 203 may be disposed in the groove, so as to carry an MCU (Micro Controller Unit) and a MEMS sensor. A mounting direction of the MEMS sensor may not be limited. For example, the MEMS sensor may be mounted facing an ultrasound emission direction as shown at the left side of FIG. 2, or the MEMS sensor may be mounted facing a side of a housing of a probe handle as shown at the right side of FIG. 2. In actual application, the MEMS sensor may be mounted facing any direction.

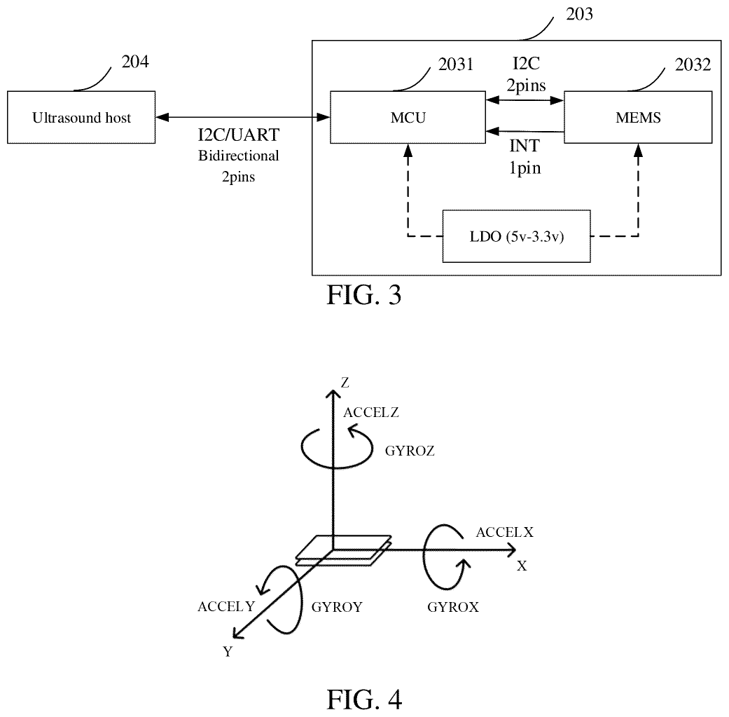

FIG. 3 is a schematic circuit diagram of an ultrasound image device. Referring to FIG. 3, an MCU 2031 and an MEMS sensor may be disposed on the carrier plate 203, and a LDO (Low Dropout Regulator) may supply a voltage to the MCU 2031 and the MEMS sensor. The carrier plate may be a circuit board configured to carry and fix electronic components, chips, or modules. The MCU 2031 and the MEMS sensor may perform bidirectional communication (2pins) by a I2C (Inter-Integrated Circuit) bus, or the MEMS sensor may send an INT (Interrupt) signal to the MCU 2031 unilaterally (1pin), and the MCU 2031 and the ultrasound host 204 may implement bidirectional communication by a I2C bus or a UART (Universal Asynchronous Receiver Transmitter) bus.

FIG. 4 is a schematic diagram of a six-axis MEMS sensor. Referring to FIG. 4, spatial three-dimensional coordinate axes X, Y, and Z may be disposed. The six-axis acceleration detected by the MEMS sensor may include three linear accelerations denoted as ACCELX, ACCELY, and ACCELZ along X, Y, and Z directions, respectively, and three rotational accelerations denoted as GYROX, GYROY, and GYROZ that rotate around X, Y, and Z, respectively. The MEMS sensor mounted on the ultrasound probe may transmit information of the detected ACCELX, ACCELY, ACCELZ, GYROX, GYROY, and GYROZ to the micro controller unit. The micro controller unit may take ACCELX, ACCELY, ACCELZ, GYROX, and GYROY, and GYROZ as the real-time acceleration of the ultrasound probe. ACCELX, ACCELY, and ACCELZ are real-time linear accelerations of the ultrasound probe, and GYROX, GYROY, and GYROZ are real-time rotational accelerations of the ultrasound probe.

Step 120 includes in a case that the real-time acceleration meets a preset condition, sending a probe activating instruction to the ultrasound host, so that the ultrasound host activates the ultrasound probe. The preset condition includes that the real-time linear acceleration is not matched with an initial linear acceleration of the ultrasound probe, and the real-time rotational acceleration exceeds a first preset threshold.

The initial linear acceleration may include a three-axis linear acceleration of the ultrasound probe at any moment in an inactive state. It should be noted that, in actual application, there is a response time for the ultrasound probe to switch from the inactive state to an active state, so that the micro controller unit may detect that the real-time acceleration meets a preset condition, and send a probe activating instruction to the ultrasound host, and the ultrasound host may activate the ultrasound probe. Since the response time is generally relatively short, the response time is ignored in the present disclosure.

The probe activating instruction may include a signal instructing the ultrasound host to activate the ultrasound probe.

The first preset threshold may include a preset threshold value of a rotational acceleration.

Alternatively, the micro controller unit may acquire the initial linear acceleration of the ultrasound probe in advance. After the real-time acceleration is detected, the real-time linear acceleration in the real-time acceleration may be compared with the initial linear acceleration, and the real-time rotational acceleration in the real-time acceleration may be compared with the first preset threshold. If the real-time linear acceleration is different from the initial linear acceleration or a difference between the real-time linear acceleration and the initial linear acceleration exceeds a second preset threshold (a preset threshold of the linear acceleration), and the real-time rotational acceleration exceeds the first preset threshold, it may indicate that the ultrasound probe moves, and the ultrasound probe needs to be activated. In this case, the micro controller unit may send a probe activating instruction to the ultrasound host. The ultrasound host may activate the ultrasound probe when receiving the probe activating instruction. Otherwise, if the difference between the real-time linear acceleration and the initial linear acceleration does not exceed the second preset threshold, or the real-time rotational acceleration does not exceed the first preset threshold, it may indicate that the ultrasound probe does not move, and the ultrasound probe does not need to be activated.

Exemplarily, the MEMS sensor may detect the six-axis acceleration of the ultrasound probe at a preset time interval. It may be set that a specified ultrasound probe of the ultrasound imaging device is in an inactive state at time t−1 (or t−2, or t−3, . . . ). The MEMS sensor may detect the six-axis acceleration. The MCU may acquire a linear acceleration including ACCELXₜ₋₁, ACCELYₜ₋₁, and ACCELₜ₋₁ in the six-axis acceleration from the MEMS sensor as the initial linear acceleration. At time t, the MEMS sensor may detect the six-axis acceleration including ACCELXₜ, ACCELYₜ, ACCELZₜ, GYROXₜ, GYROYₜ, and GYROZₜ. The MCU may acquire a linear acceleration including ACCELXₜ, ACCELYₜ, and ACCELZₜ from the MEMS sensor as the real-time linear acceleration, and acquire a rotational acceleration including GYROXₜ, GYROYₜ, and GYROZₜ from the MEMS sensor as the real-time rotational acceleration. The first preset threshold T1 may be preset, and the second preset threshold T2 may be preset. If any one component of the linear acceleration changes, for example, any one of the following relationships is satisfied: |ACCELXₜ₋₁-ACCELXₜ|>T2, |ACCELYₜ₋₁-ACCELYₜ|>T2, or |ACCELZₜ₋₁-ACCELZₜ|>T2, and any one component of the real-time rotational acceleration exceeds the preset threshold, for example, any one of the following relationships is satisfied: |GYROXₜ|>T1, |GYROYₜ|>T1, or |GYROZₜ|>T1, it may indicate that the ultrasound probe moves, and the MCU may send the probe activating instruction to the ultrasound host. Otherwise, if components of the linear acceleration do not change, for example, the following relationships are satisfied: |ACCELXₜ₋₁-ACCELXₜ|≤T2, |ACCELYₜ₋₁-ACCELYₜ|≤T2, and |ACCELZₜ₋₁-ACCELZₜ|≤T2, or components of the real-time rotational acceleration do not exceed the preset threshold, for example, the following relationships are satisfied: |GYROXₜ|≤T1, |GYROYₜ|≤T1, and |GYROZₜ|≤T1, it may indicate that the ultrasound probe does not move and does not need to be activated. In this case, the MCU may take ACCELXₜ, ACCELYₜ, and ACCELZₜ as a new initial linear acceleration, and acquire a new real-time linear acceleration including ACCELXₜ₊₁, ACCELYₜ₊₁, and ACCELZₜ₊₁, and a new real-time rotational acceleration including GYROXₜ₊₁, GYROYₜ₊₁, and GYROZₜ₊₁ from the MEMS sensor. The MCU may repeat the foregoing process until the probe activating instruction is sent to the ultrasound host.

In the foregoing ultrasound probe activating method, the real-time acceleration of the ultrasound probe detected by the motion sensor is acquired. The real-time acceleration includes the real-time linear acceleration and the real-time rotational acceleration. When the real-time acceleration meets the preset condition, the probe activating instruction is sent to the ultrasound host, so that the ultrasound host activates the ultrasound probe. The preset condition includes that the real-time linear acceleration is not matched with the initial linear acceleration of the ultrasound probe, and the real-time rotational acceleration exceeds the first preset threshold. The real-time acceleration when the ultrasound probe is picked up is used to trigger the ultrasound host to activate the ultrasound probe. In other words, when the real-time linear acceleration of the ultrasound probe changes and the real-time rotational acceleration exceeds the first preset threshold, the ultrasound host is automatically controlled to activate the ultrasound probe, so that no manual activation of the ultrasound probe is required, thereby increasing convenience of using the ultrasound imaging device.

In an exemplarily embodiment, the real-time linear acceleration may include three real-time linear components and the real-time rotational acceleration may include three real-time rotational components. The preset condition may further include that any one of the three real-time linear components of the real-time linear acceleration is not matched with a corresponding initial linear component, and any one of the three real-time rotational components of the real-time rotational acceleration exceeds the first preset threshold.

The three real-time linear components may be components of the real-time linear acceleration. The three real-time rotational components may be components of the real-time rotational acceleration. The corresponding initial linear component may be a component of the initial linear acceleration.

Alternatively, each component of the real-time linear acceleration may be taken as a real-time linear component, each component of the initial linear acceleration may be taken as the corresponding initial linear component, and each component of the real-time rotational acceleration may be taken as a real-time rotation component. Furthermore, the preset condition may be set that any real-time linear component is not matched with the corresponding initial linear component, and any real-time rotational component exceeds the first preset threshold. The real-time linear component is not matched with the corresponding initial linear component, it means that the real-time linear component is different from the corresponding initial linear component or a difference between the real-time linear component and the corresponding initial linear component exceeds the second preset threshold.

Exemplarily, three initial linear components denoted as ACCELXₜ₋₁, ACCELYₜ₋₁, and ACCELZₜ₋₁, and three real-time linear components denoted as ACCELXₜ, ACCELYₜ, and ACCELZₜ may be obtained by reading a linear acceleration register of the MEMS sensor, and three real-time rotational components denoted as GYROXₜ, GYROYₜ, and GYROZₜ may be obtained by reading the rotational acceleration register of the MEMS sensor. The first preset threshold T1 may be preset, and the second preset threshold T2 may be preset. If any one of the following relationships is satisfied: |ACCELXₜ₋₁-ACCELX: |>T2, |ACCELYₜ₋₁-ACCELYₜ|>T2, or |ACCELZₜ₋₁-ACCELZₜ|>T2, and any one of the following relationships is satisfied: |GYROXₜ|>T1, |GYROYₜ|>T1, or |GYROZₜ|>T1, it may indicate that the ultrasound probe moves, and the MCU may send the probe activating instruction to the ultrasound host.

Otherwise, if the following relationships are satisfied: |ACCELXₜ₋₁-ACCELXₜ|≤T2, |ACCELYₜ₋₁-ACCELYₜ|≤T2, and |ACCELZₜ₋₁-ACCELZₜ|≤T2, or the following relationships are satisfied: |GYROXₜ|≤T1, |GYROYₜ|≤T1, and |GYROZₜ|≤T1, it may indicate that the ultrasound probe does not move and does not need to be activated.

In the present embodiment, the real-time linear acceleration may include three real-time linear components, the real-time rotational acceleration may include three real-time rotational components, the preset condition further includes that any one of the three real-time linear components of the real-time linear acceleration is not matched with the corresponding initial linear component, and any one of the three real-time rotational components of the real-time rotational acceleration exceeds the first preset threshold. In this way, the real-time acceleration may be fully used to automatically determine whether the ultrasound probe needs to be activated, thereby improving convenience of using the ultrasound imaging device.

In an exemplarily embodiment, the step 120 may specifically include: when a difference between any one of the three real-time linear components of the real-time linear acceleration and the corresponding initial linear component exceeds a second preset threshold, comparing each of the three real-time rotational components of the real-time rotational acceleration with the first preset threshold; when any one of the three real-time rotational components of the real-time rotational acceleration exceeds the first preset threshold, sending the probe activating instruction to the ultrasound host.

The second preset threshold may be a preset threshold of the linear acceleration.

Alternatively, the micro controller unit may compare each real-time linear component of the real-time linear acceleration with the corresponding initial linear component, respectively. If a difference between any one real-time linear component and the corresponding initial linear component exceeds the second preset threshold, the micro controller unit may compare each real-time rotational component of the real-time rotational acceleration with the first preset threshold. If any one real-time rotational component exceeds the first preset threshold, the micro controller unit may send the probe activating instruction to the ultrasound host. Otherwise, if the difference between each real-time linear component and the corresponding initial linear component does not exceed the second preset threshold, or each real-time rotational component does not exceed the first preset threshold, the micro controller unit may not need to send the probe activating instruction to the ultrasound host.

Exemplarily, the first preset threshold T1 may be preset, and the second preset threshold T2 may be preset. The three real-time linear components ACCELXₜ, ACCELYₜ, and ACCELZₜ may be compared with corresponding initial linear components ACCELXₜ₋₁, ACCELYₜ₋₁, and ACCELZₜ₋₁, respectively. If any one of the following relationships is satisfied: |ACCELXₜ₋₁-ACCELXₜ|>T2, |ACCELYₜ₋₁-ACCELYₜ|>T2, or |ACCELZₜ₋₁-ACCELZₜ|>T2, the three real-time rotational components GYROXₜ, GYROYₜ, and GYROZₜ may be compared with the first preset threshold T1. If any one of the following relationships is satisfied: |GYROXₜ|>T1, |GYROYₜ|>T1, or |GYROZₜ|>T1, the probe activating instruction may be sent to the ultrasound host.

In the present embodiment, when the difference between any one of the three real-time linear components of the real-time linear acceleration and the corresponding initial linear component exceeds a second preset threshold, each of the three real-time rotational components of the real-time rotational acceleration may be compared with the first preset threshold. When any one of the three real-time rotational components of the real-time rotational acceleration exceeds the first preset threshold, the probe activating instruction may be sent to the ultrasound host. When it is detected that the ultrasound probe moves, it is possible to further detect whether the ultrasound probe rotates, so as to avoid environmental vibration or artificial touch causing false activation of the ultrasound probe, and increase reliability of activation of the ultrasound probe.

In an exemplarily embodiment, the step 120 may specifically include: in the case that the real-time acceleration meets the preset condition, updating an original displacement count value to obtain an updated displacement count value, sending the updated displacement count value to the ultrasound host, and activating, by the ultrasound host, the ultrasound probe according to the updated displacement count value.

The original displacement count value may be an original displacement count value in the displacement counter. The updated displacement count value may be an updated displacement count value in the displacement counter. The displacement counter may be a counter that records the quantity of times of displacement.

Alternatively, the displacement counter may be disposed in the micro controller unit, and the displacement counter may store the original displacement count value at a previous moment. When the real-time linear acceleration is not matched with the initial linear acceleration, and the real-time rotational acceleration exceeds the first preset threshold, the original displacement count value may be increased by a preset value to obtain the updated displacement count value. The micro controller unit may send the updated displacement count value to the ultrasound host. The original displacement count value at the previous moment may be recorded in the ultrasound host, and a received displacement count value may be compared with the original displacement count value at the previous moment. If the displacement count value is increased, the ultrasound probe is activated. Otherwise, if the real-time linear acceleration is matched with the initial linear acceleration, or the real-time rotational acceleration does not exceed the first preset threshold, the micro controller unit may send the original displacement count value to the ultrasound host. In this case, the displacement count value may not be increased, and the ultrasound probe may not be activated.

Exemplarily, at time t−1, both the MCU and the ultrasound host may record the original displacement count value denoted as count. At time t, if any one of the following relationships is satisfied: |ACCELXₜ₋₁-ACCELXₜ|>T2, |ACCELYₜ₋₁-ACCELYₜ|>T2, or |ACCELZₜ₋₁-ACCELZₜ|>T2, and any one of the following relationships is satisfied: |GYROXₜ|>T1, |GYROYₜ|>T1, or |GYROZₜ|>T1, the MCU may increase the original displacement count value of the displacement counter by 1 to obtain the updated displacement count value denoted as count+1, and send the updated displacement count value count+1 to the ultrasound host. The ultrasound host may recognize that the received count+1 increases compared with the previous recorded original displacement count value count, and then activate the ultrasound probe. Otherwise, at time t, if the following relationships are satisfied: |ACCELXₜ₋₁-ACCELXₜ| ≤T2, |ACCELYₜ₋₁-ACCELYₜ|≤T2, and |ACCELZₜ₋₁-ACCELZₜ|≤T2, or the following relationships are satisfied: |GYROXₜ|≤T1, |GYROYₜ|≤T1, and |GYROZₜ|≤T1, the MCU may send the original displacement count value count to the ultrasound host. The ultrasound host may recognize that the received count does not increase compared with the previous recorded original displacement count value count, and then not activate the ultrasound probe. In this way, the ultrasound host may determine, by cyclically querying whether a value of the displacement counter changes, whether the probe needs to be activated. When the ultrasound host finds that the value of the displacement counter changes, the ultrasound probe may be activated.

In the present embodiment, in the case that the real-time acceleration meets the preset condition, the original displacement count value may be updated to obtain the updated displacement count value, the updated displacement count value may be sent to the ultrasound host, and the ultrasound host may activate the ultrasound probe according to the updated displacement count value. In this way, when it is detected that the real-time acceleration of the ultrasound probe meets the preset condition when the ultrasound probe is picked up, the micro controller unit may send the displacement count value to the ultrasound host, and the ultrasound host may determine, by detecting the displacement count value, whether the ultrasound probe needs to be activated, thereby improving activating efficiency of the ultrasound probe.

In an exemplarily embodiment, before the step 110, the method may further include: reading an initial acceleration of the ultrasound probe from a register of the motion sensor in response to a received interrupt instruction, and extracting the initial linear acceleration of the ultrasound probe from the initial acceleration. The interrupt instruction may be sent by the motion sensor to the micro controller unit when the motion sensor perceives a gravity acceleration of the ultrasound probe.

The interrupt instruction may be a signal that is sent by the motion sensor to the micro controller unit and that instructs the micro controller unit to execute an interrupt operation.

The initial acceleration may be a six-axis acceleration of the ultrasound probe at a moment in an inactive state.

Alternatively, the motion sensor may perceive the six-axis acceleration of the ultrasound probe in real time, and store information of the six-axis acceleration of the ultrasound probe in the register of the motion sensor. When the motion sensor perceives the gravity acceleration, the motion sensor may send the interrupt instruction to the micro controller unit. When receiving the interrupt instruction, the micro controller unit may read information of the six-axis acceleration from the register of the motion sensor, take the six-axis acceleration as the initial acceleration of the ultrasound probe, extract the three-axis linear acceleration from the initial acceleration, and take the three-axis linear acceleration as the initial linear acceleration of the ultrasound probe.

Exemplarily, when the MEMS sensor perceives a gravity acceleration, the MEMS sensor may send an INT interrupt signal to the MCU. In response to the received INT interrupt signal, the MCU may read a register of the MEMS sensor to obtain the six-axis acceleration including ACCELXₜ₋₁, ACCELYₜ₋₁, ACCELZₜ₋₁, GYROXₜ₋₁, GYROYₜ₋₁, and GYROZₜ₋₁ of the ultrasound probe at time t−1 stored in the register, and take ACCELXₜ₋₁, ACCELYₜ₋₁, and ACCELZₜ₋₁ as the initial linear acceleration of the ultrasound probe.

In the present embodiment, the initial acceleration of the ultrasound probe may be read from the register of the motion sensor in response to the received interrupt instruction, and the initial linear acceleration of the ultrasound probe may be extracted from the initial acceleration. The interrupt instruction may trigger the micro controller unit to acquire the initial linear acceleration of the ultrasound probe, so as to ensure execution of the ultrasound probe activating method.

In an exemplary embodiment, the step 110 may specifically include: reading a register of the motion sensor in real time to obtain the real-time acceleration of the ultrasound probe.

Alternatively, the motion sensor may perceive the six-axis acceleration of the ultrasound probe in real time, store the information of the detected six-axis acceleration in the register. The micro controller unit may read the register of the motion sensor in real time, and take the read six-axis acceleration as the real-time acceleration of the ultrasound probe.

Exemplarily, at time t, the MEMS sensor may collect the six-axis acceleration including ACCELXₜ, ACCELYₜ, ACCELZₜ, GYROXₜ, GYROYₜ, and GYROZₜ, and store the six-axis acceleration including ACCELXₜ, ACCELYₜ, ACCELZₜ, GYROXₜ, GYROYₜ, and GYROZₜ in the register. The MCU may read ACCELXₜ, ACCELYₜ, ACCELZₜ, GYROXₜ, GYROYₜ, and GYROZₜ from the register of the MEMS sensor as the real-time acceleration of the ultrasound probe.

In this embodiment, the real-time acceleration of the ultrasound probe may be obtained by reading the register of the motion sensor in real time, so that the real-time acceleration of the ultrasound probe may be quickly determined by the motion sensor, thereby improving activating efficiency of the ultrasound probe.

To facilitate a deeper understanding of the present disclosure by one skilled in the art, the following will be illustrated with a specific example.

An automatic activating detection method of an ultrasound probe that is picked up based on an MEMS sensor is provided in the present disclosure. The method may require integrating a six-axis MEMS sensor inside the ultrasound probe. Referring to FIG. 4, six-axis sensing data provided by the six-axis MEMS sensor may include: acceleration data including ACCELX, ACCELY, and ACCELZ along the X, Y, and Z axes, and the rotational acceleration data including GYROX, GYROY, GYROZ around the X, Y, and Z axes. Referring to FIG. 2, the MEMS sensor may be mounted in an ultrasound probe cavity and a circuit design may refer to FIG. 3.

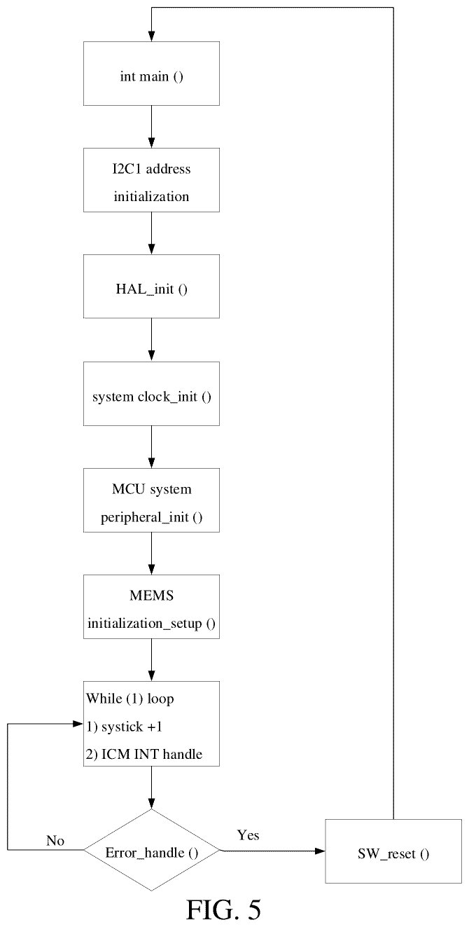

FIG. 5 is a flowchart of an MCU firmware program. Referring to FIG. 5, the I2C, a HAL (Hardware Abstraction Layer), a system clock, an MCU peripherals, the MEMS sensor, etc. may be initialized first before entering a loop. A system count denoted as systick may be added by 1, and a probe pickup detecting program block denoted as ICM INT handle may be executed, during which a detecting program block denoted as error_handle may be used to determine whether there is an error in the program execution of ICM INT handle, and if yes, the system is reset (SW_reset) and initialized again.

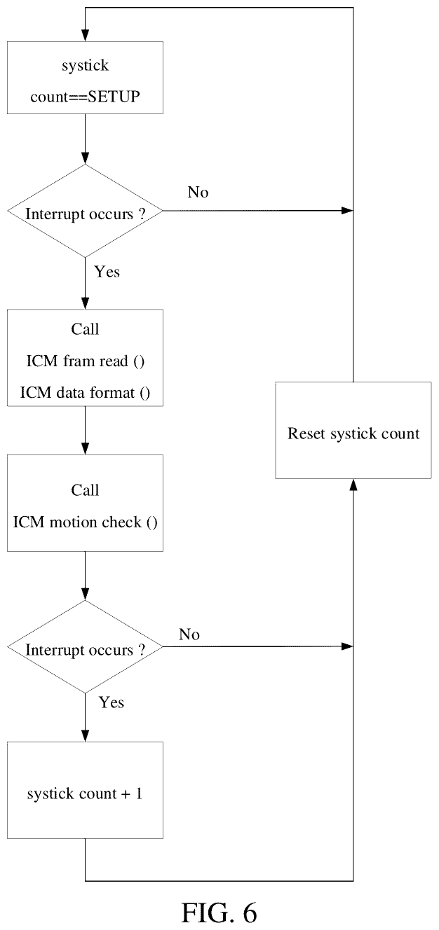

In the detecting program block ICM INT handle, the MEMS sensor may be set to send the INT interrupt signal to the MCU when any axis perceives 1g (1 gravity acceleration). FIG. 6 is a flowchart of a probe pickup detecting program. Referring to FIG. 6, the detecting program block ICM INT handle may specifically include the following step 1 to step 4.

Step 1 may include detecting whether the INT interrupt occurs. When any axis of the MEMS sensor detects 1g, the INT interrupt signal may be sent. Due to the earth's gravity, at least one axis may always detect 1g. At this moment, data may be read from a register of the MEMS sensor by ICM frame read( ) and ICM data format( ).

Step 2 may include after the MCU receives the INT interrupt signal, determining whether the ultrasound probe moves along the X/Y/Z axis by ICM motion check( ) (in the INT handle, determining whether the ultrasound probe moves by reading an ACCEL (Linear Acceleration) register value of the MEMS sensor), if yes (i.e., when the INT interrupt occurs, the axis for which the ACCEL is validly output is not the same as the last axis in the record), determining that the MEMS sensor has moved, and thus determining that an action has occurred.

At step 3, for a reciprocating motion along on a single axis, for example, a strict round-trip movement of X-X, Y-Y, or Z-Z, auxiliary determination of GYRO (rotational acceleration) data may be added. Due to a transient of the rotational acceleration (i.e., data may be read only when rotation occurs), when the INT interrupt is triggered, the MCU may simultaneously read data of the GYROX/Y/Z-axis gyroscope. If data of any axis of the gyroscope is greater than a preset threshold (e.g., 50 dps), it may be determined in this case that a displacement occurs on the MEMS sensor.

At step 4, when the displacement of the MEMS sensor is confirmed, the MCU may add the system count denoted as systick count by 1 to represent that an action is detected. A host program may determine, according to whether a value of the displacement counter is increased, whether a picked-up action is performed on the probe on which the MEMS sensor is attached.

The foregoing automatic activating detection method of the ultrasound probe that is picked up based on the MEMS sensor is provided in the present embodiment. For parameter setting, measurement range selection, numerical sampling, and mounting mode of the MEMS sensor in the probe, a detailed solution may be provided. The MEMS sensor configured on the probe may not be required to have a specific mounting position and mounting angle. In some embodiments, in the foregoing ultrasound probe activating method, the user may not need to perform a special action (e.g., double-tap a special position of the probe or intentionally shake the probe) to activate the probe, thereby avoiding abrasion at the specific position of the probe or premature cable failure. In some embodiments, the foregoing ultrasound probe activating method may provide a sampling time of an output value of the MEMS sensor and a corresponding processing method. When the linear acceleration changes and the rotational acceleration is detected, the probe activating instruction may be sent to the ultrasound host, thereby effectively implementing a probe activating function. In some embodiments, the foregoing ultrasound probe activating method may effectively detect an action in which the ultrasound probe is picked up, and trigger a probe activating action of the system in a timely and effective manner, thereby optimizing an operation procedure of ultrasound inspection. In some embodiments, in the foregoing ultrasound probe activating method, automatic activation of the ultrasound probe may be implemented, and no doctor/technician may need to operate a panel button separately, thereby improving available performance of a medical ultrasound imaging host.

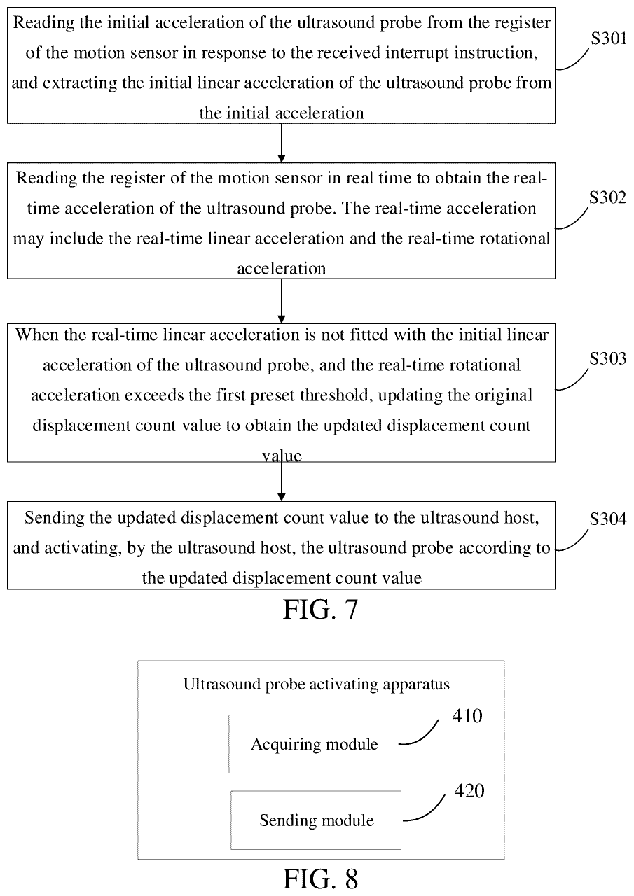

In an embodiment, referring to FIG. 7, an ultrasound probe activating method is provided in the present disclosure. Taking that the method is applied to a micro controller unit as an example, the method may include the following step 301 to step 304.

Step 301 may include reading the initial acceleration of the ultrasound probe from the register of the motion sensor in response to the received interrupt instruction, and extracting the initial linear acceleration of the ultrasound probe from the initial acceleration. The interrupt instruction may be sent by the motion sensor to the micro controller unit when the motion sensor perceives the gravity acceleration of the ultrasound probe.

Step 302 may include reading the register of the motion sensor in real time to obtain the real-time acceleration of the ultrasound probe. The real-time acceleration may include the real-time linear acceleration and the real-time rotational acceleration.

Step 303 may include when the real-time linear acceleration is not matched with the initial linear acceleration of the ultrasound probe, and the real-time rotational acceleration exceeds the first preset threshold, updating the original displacement count value to obtain the updated displacement count value.

Step 304 may include sending the updated displacement count value to the ultrasound host, and activating, by the ultrasound host, the ultrasound probe according to the updated displacement count value.

Alternatively, the motion sensor may send the interrupt instruction to the micro controller unit when at least one gravity acceleration is detected. When receiving the interrupt instruction, the micro controller unit may read the initial acceleration of the ultrasound probe from the register of the motion sensor, and extract the three-axis linear acceleration from the initial acceleration as the initial linear acceleration. The micro controller unit may further read the real-time acceleration of the ultrasound probe from the register of the motion sensor. The real-time acceleration may include a currently detected three-axis linear acceleration and a currently detected three-axis rotational acceleration, i.e., the real-time linear acceleration and the real-time rotational acceleration. If the micro controller unit detects that the difference between any component of the real-time linear acceleration and the corresponding component of the initial linear acceleration exceeds the second preset threshold, and any component of the real-time rotational acceleration exceeds the first preset threshold, the displacement count value in the displacement counter may be increased by 1 to obtain the updated displacement count value, and the updated displacement count value may be sent to the ultrasound host. In this case, the updated displacement count value may be taken as the probe activating instruction. When the ultrasound host detects that the displacement count value is changed, the ultrasound sensor may be activated. Otherwise, if the difference between each component of the real-time linear acceleration and the corresponding component of the initial linear acceleration does not exceed the second preset threshold, or each component of the real-time rotational acceleration does not exceed the first preset threshold, the ultrasound sensor may not need to be activated. In this case, the real-time acceleration may be taken as the initial acceleration, and step 302 to step 304 may be repeated.

In the present embodiment, the real-time acceleration when the ultrasound probe is picked up is used to trigger the ultrasound host to activate the ultrasound probe. In other words, when the real-time linear acceleration of the ultrasound probe changes and the real-time rotational acceleration exceeds the first preset threshold, the ultrasound host is automatically controlled to activate the ultrasound probe, so that no manual activation of the ultrasound probe is required, thereby increasing convenience of using the ultrasound imaging device.

It should be understood that, although steps in the flowchart related to the foregoing embodiments are sequentially displayed according to an instruction of an arrow, these steps are not necessarily sequentially performed according to the instruction of the arrow. Unless expressly stated in this specification, these steps are not performed in a strict order, and these steps may be performed in another order. In addition, at least a part of steps in the flowchart involved in the foregoing embodiments may include multiple steps or multiple phases. These steps or phases are not necessarily performed at a same moment, but may be performed at different moments. These steps or phases are not necessarily performed sequentially, but may be performed alternately or alternately with another step or at least a part of steps or phases in another step.

Based on the same invention concept, an ultrasound probe activating apparatus configured to implement the foregoing involved ultrasound probe activating method is further provided in an embodiment of the present disclosure. An implementation solution provided by the apparatus is similar to the implementation solution described in the foregoing method. Therefore, a specific limitation in one or more embodiments of the ultrasound probe activating apparatus provided below may refer to the foregoing limitation in the ultrasound probe activating method. Details are not described herein again.

In an exemplary embodiment, referring to FIG. 8, an ultrasound probe activating apparatus is provided. The apparatus is applied to a micro controller unit. The micro controller unit is disposed on an ultrasound probe of an ultrasound imaging device, a motion sensor is further disposed on the ultrasound probe, the micro controller unit is connected to the motion sensor and an ultrasound host of the ultrasound imaging device, respectively, and the apparatus includes an acquiring module 410 and a sending module 420.

The acquiring module 410 is configured for acquiring a real-time acceleration of the ultrasound probe detected by the motion sensor. The real-time acceleration includes a real-time linear acceleration and a real-time rotational acceleration.

The sending module 420 is configured for sending a probe activating instruction to the ultrasound host in a case that the real-time acceleration meets a preset condition, so that the ultrasound host activates the ultrasound probe. The preset condition includes that the real-time linear acceleration is not matched with an initial linear acceleration of the ultrasound probe, and the real-time rotational acceleration exceeds a first preset threshold.

In an exemplarily embodiment, the real-time linear acceleration may include three real-time linear components and the real-time rotational acceleration includes three real-time rotational components. The preset condition may further include that any one of the three real-time linear components of the real-time linear acceleration is not matched with a corresponding initial linear component, and any one of the three real-time rotational components of the real-time rotational acceleration exceeds the first preset threshold.

In an exemplarily embodiment, the sending module 420 is further configured for comparing each of the three real-time rotational components of the real-time rotational acceleration with the first preset threshold when a difference between any one of the three real-time linear components of the real-time linear acceleration and the corresponding initial linear component exceeds a second preset threshold, and sending the probe activating instruction to the ultrasound host when any one of the three real-time rotational components of the real-time rotational acceleration exceeds the first preset threshold.

In an exemplarily embodiment, the sending module 420 is further configured for updating an original displacement count value to obtain an updated displacement count value in the case that the real-time acceleration meets the preset condition, sending the updated displacement count value to the ultrasound host, and activating, by the ultrasound host, the ultrasound probe according to the updated displacement count value.

In an exemplarily embodiment, the ultrasound probe activating apparatus may further include an interrupt responding module, which is configured for reading an initial acceleration of the ultrasound probe from a register of the motion sensor in response to a received interrupt instruction, and extracting the initial linear acceleration of the ultrasound probe from the initial acceleration. The interrupt instruction may be sent by the motion sensor to the micro controller unit when the motion sensor perceives a gravity acceleration of the ultrasound probe.

In an exemplarily embodiment, the acquiring module 410 is further configured for reading a register of the motion sensor in real time to obtain the real-time acceleration of the ultrasound probe.

All modules in the foregoing ultrasound probe activating apparatus may be implemented in whole or in part by software, hardware, and a combination thereof. The foregoing modules may be embedded in or independent of a processor in the micro controller unit in a hardware form, or may be stored in a memory in the micro controller unit in a software form, so that the processor may invoke to execute an operation corresponding to the foregoing modules.

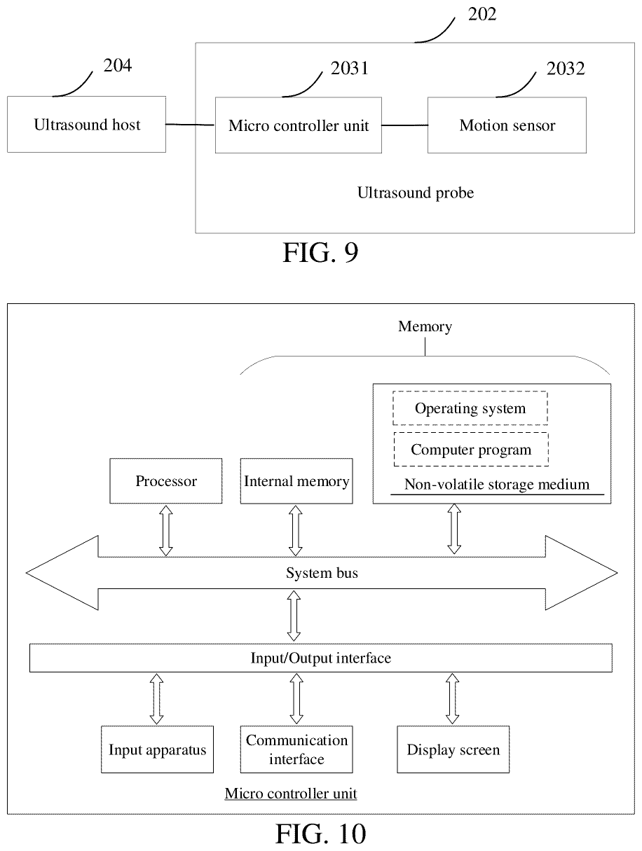

In an exemplarily embodiment, referring to FIG. 9, an ultrasound imaging device is further provided in the present disclosure, including an ultrasound host 204 and an ultrasound probe 202. A micro controller unit 2031 and a motion sensor 2032 are disposed on the ultrasound probe 202, the micro controller unit 2031 is connected to the motion sensor 2032 and the ultrasound host 204, respectively.

The motion sensor 2032 is configured for perceiving a real-time acceleration of the ultrasound probe 202 and sending information of the real-time acceleration to the micro controller unit 2031, and the real-time acceleration includes a real-time linear acceleration and a real-time rotational acceleration.

The micro controller unit 2031 is configured for sending a probe activating instruction to the ultrasound host 204 in a case that the real-time acceleration meets a preset condition. The preset condition includes that the real-time linear acceleration is not matched with an initial linear acceleration of the ultrasound probe, and the real-time rotational acceleration exceeds a first preset threshold.

The ultrasound host 204 is configured for activating the ultrasound probe 202 when receiving the probe activating instruction.

Alternatively, the motion sensor may take the six-axis acceleration of the ultrasound probe detected in real time as the real-time acceleration of the ultrasound probe, and send information of the real-time acceleration to the micro controller unit. The micro controller unit may compare the real-time linear acceleration in the real-time acceleration with the initial linear acceleration of the ultrasound probe, and compare the real-time rotational acceleration in the real-time acceleration with the first preset threshold. When the difference between the real-time linear acceleration and the initial linear acceleration exceeds the second preset threshold, and the real-time rotational acceleration exceeds the first preset threshold, the displacement count value may be added by 1 as the probe activating instruction to send to the ultrasound host. The ultrasound host may compare the received displacement count value and the previously stored displacement count value, and activate the ultrasound probe when detecting that the displacement count value increases.

Since specific processing processes of the motion sensor, the micro controller unit, and the ultrasound host is described in detail in the foregoing embodiment, details are not described herein again.

In the foregoing ultrasound imaging device, the real-time acceleration of the ultrasound probe is used to trigger the ultrasound host to activate the ultrasound probe when the ultrasound probe is picked up. In other words, when the real-time linear acceleration of the ultrasound probe changes and the real-time rotational acceleration exceeds the first preset threshold, the ultrasound host is automatically controlled to activate the ultrasound probe, so that no manual activation of the ultrasound probe is required, thereby increasing convenience of using the ultrasound imaging device.

In an exemplarily embodiment, a micro controller unit is further provided, the micro controller unit may be a terminal, and an internal structure diagram of the micro controller unit may refer to FIG. 10. The micro controller unit may include a processor, a memory, an input/output interface, a communication interface, a display unit, and an input apparatus. The processor, the memory, and the input/output interface may be connected by the system bus, and the communication interface, the display unit, and the input apparatus may be connected to the system bus by the input/output interface. The processor of the computer device is configured to provide a computing and control capability. The memory of the micro controller unit may include a non-volatile storage medium and an internal memory. The non-volatile storage medium may store an operating system and a computer program. The internal storage may provide an environment for running the operating system and the computer program in the non-volatile storage medium. The input/output interface of the computer device is configured to exchange information between the processor and an external device. The communication interface of the micro controller unit is configured to communicate with an external terminal in a wired or wireless manner. The wireless manner may be implemented by a WIFI, a mobile cellular network, an NFC (near field communication), or other technology. The computer program is executed by the processor to implement the ultrasound probe activating method. The display unit of the micro controller unit is configured to form a visual picture, which may be a display screen, a projection apparatus, or a virtual reality imaging apparatus. The display screen may be a liquid crystal display screen or an electronic ink display screen. The input apparatus of the micro controller unit may be a touch layer covered on the display screen, may be a key, a trackball, or a touchpad disposed on a housing of the micro controller unit, or may be an external keyboard, a touchpad, a mouse, or the like.

One skilled in the art may understand that the structure shown in FIG. 10 is merely a block diagram of some structures related to the solutions of the present disclosure, and does not constitute a limitation on the micro controller unit to which the solutions of the present disclosure are applied. A specific micro controller unit may include more or fewer members than those shown in the figure, or combine some members, or have different member arrangements.

In an exemplary embodiment, a micro controller unit is further provided in the present disclosure, including a memory and a processor. A computer program is stored in the memory, and the processor is configured to execute the computer program to implement the steps in the foregoing method embodiments.

In an exemplary embodiment, a computer-readable storage medium is further provided, which stores a computer program. The computer program is executed by a processor to implement the steps in the foregoing method embodiments.

In an exemplary embodiment, a computer program product is further provided, which includes a computer program. The computer program is executed by a processor to implement the steps in the foregoing method embodiments.

It should be noted that user information (including but not limited to user device information, user personal information, and the like) and data (including but not limited to data used for analysis, stored data, and displayed data) involved in the present disclosure are information and data that are authorized by the user or that are fully authorized by each party, and collection, use, and processing of related data need to comply with related regulations.

One skilled in the art may understand that all or a part of the processes in the methods in the foregoing embodiments may be implemented by a computer program instructing related hardware. The computer program may be stored in a non-volatile computer readable storage medium. When the computer program is executed, the processes in the foregoing methods embodiments may be included. Any reference to a memory, a database, or other medium used in the embodiments provided in the present disclosure may include at least one of a non-volatile memory or a volatile memory. The non-volatile memory may include a Read-Only Memory (ROM), a magnetic tape, a floppy disk, a flash memory, an optical memory, a high-density embedded non-volatile memory, a Resistive Random Access Memory (ReRAM), a Magneto resistive Random Access Memory (MRAM), a Ferroelectric Random Access Memory (FRAM), a Phase Change Memory (PCM), a graphene memory, and the like. The volatile memory may include a Random Access Memory (RAM), an external cache, or the like. As an illustration and not a limitation, the RAM may be in multiple forms, such as a Static Random Access Memory (SRAM) or a Dynamic Random Access Memory (DRAM). The databases involved in the embodiments provided in the present disclosure may include at least one of a relational database or a non-relational database. The non-relational database may include a distributed database based on a block chain or the like, which is not limited thereto. The processor in the embodiments provided in present disclosure may be a general-purpose processor, a central processing unit, a graphics processor, a digital signal processor, a programmable logic device, a quantum computing-based data processing logic device, or the like, which is not limited thereto.

All the technical features in the foregoing embodiments may be any combination. To make the description brief, all possible combinations of the technical features in the foregoing embodiments are not described. However, as long as there is no contradiction between the combinations of the technical features, it should be considered as the scope described in this specification.

The foregoing embodiments represent only several implementation manners of the present disclosure, and descriptions thereof are relatively specific and detailed, but may not be construed as a limitation on the scope of the present disclosure. It should be noted that one skilled in the art may make some modifications and improvements without departing from the concept of the present disclosure, which are within the protection scope of the present disclosure. Therefore, the protection scope of the present disclosure shall be subject to the attached claims.

What is claimed is:

1. An ultrasound probe activating method, comprising:
acquiring a real-time acceleration of an ultrasound probe detected by a motion sensor, wherein the motion sensor is disposed on the ultrasound probe, and the real-time acceleration comprises a real-time linear acceleration and a real-time rotational acceleration; and
in a case that the real-time acceleration meets a preset condition, sending a probe activating instruction to an ultrasound host, so that the ultrasound host activates the ultrasound probe, wherein the preset condition comprises that the real-time linear acceleration is not matched with an initial linear acceleration of the ultrasound probe, and the real-time rotational acceleration exceeds a first preset threshold.

2. The method of claim 1, wherein the real-time linear acceleration comprises three real-time linear components and the real-time rotational acceleration comprises three real-time rotational components; the preset condition further comprises that any one of the three real-time linear components of the real-time linear acceleration is not matched with a corresponding initial linear component, and any one of the three real-time rotational components of the real-time rotational acceleration exceeds the first preset threshold.

3. The method of claim 2, wherein in the case that the real-time acceleration meets the preset condition, sending the probe activating instruction to the ultrasound host further comprises:
when a difference between any one of the three real-time linear components of the real-time linear acceleration and the corresponding initial linear component exceeds a second preset threshold, comparing each of the three real-time rotational components of the real-time rotational acceleration with the first preset threshold; and
when any one of the three real-time rotational components of the real-time rotational acceleration exceeds the first preset threshold, sending the probe activating instruction to the ultrasound host.

4. The method of claim 1, wherein in the case that the real-time acceleration meets the preset condition, sending the probe activating instruction to the ultrasound host further comprises:
in the case that the real-time acceleration meets the preset condition, updating an original displacement count value to obtain an updated displacement count value; and
sending the updated displacement count value to the ultrasound host, and activating, by the ultrasound host, the ultrasound probe according to the updated displacement count value.

5. The method of claim 1, wherein updating the original displacement count value comprises increasing the original displacement count value by a preset value.

6. The method of claim 1, wherein before acquiring the real-time acceleration of the ultrasound probe detected by the motion sensor, the method further comprises:
reading an initial acceleration of the ultrasound probe from a register of the motion sensor in response to a received interrupt instruction, wherein the interrupt instruction is sent by the motion sensor when the motion sensor perceives a gravity acceleration of the ultrasound probe; and
extracting the initial linear acceleration of the ultrasound probe from the initial acceleration.

7. The method of claim 1, wherein acquiring the real-time acceleration of the ultrasound probe detected by the motion sensor further comprises:
reading a register of the motion sensor in real time to obtain the real-time acceleration of the ultrasound probe.

8. An ultrasound probe activating apparatus, comprising:
means for acquiring a real-time acceleration of an ultrasound probe detected by a motion sensor, wherein the motion sensor is disposed on the ultrasound probe, and the real-time acceleration comprises a real-time linear acceleration and a real-time rotational acceleration; and
means for sending a probe activating instruction to an ultrasound host in a case that the real-time acceleration meets a preset condition, so that the ultrasound host activates the ultrasound probe, wherein the preset condition comprises that the real-time linear acceleration is not matched with an initial linear acceleration of the ultrasound probe, and the real-time rotational acceleration exceeds a first preset threshold.

9. An ultrasound imaging device, comprising an ultrasound host and an ultrasound probe, wherein a micro controller unit and a motion sensor are disposed on the ultrasound probe, the micro controller unit is connected to the motion sensor and the ultrasound host, respectively;
the motion sensor is configured for perceiving a real-time acceleration of the ultrasound probe and sending information of the real-time acceleration to the micro controller unit, wherein the real-time acceleration comprises a real-time linear acceleration and a real-time rotational acceleration;
the micro controller unit is configured for sending a probe activating instruction to the ultrasound host in a case that the real-time acceleration meets a preset condition, wherein the preset condition comprises that the real-time linear acceleration is not matched with an initial linear acceleration of the ultrasound probe, and the real-time rotational acceleration exceeds a first preset threshold; and
the ultrasound host is configured for activating the ultrasound probe when receiving the probe activating instruction.

10. The ultrasound imaging device of claim 9, wherein the real-time linear acceleration comprises three real-time linear components and the real-time rotational acceleration comprises three real-time rotational components; the preset condition further comprises that any one of the three real-time linear components of the real-time linear acceleration is not matched with a corresponding initial linear component, and any one of the three real-time rotational components of the real-time rotational acceleration exceeds the first preset threshold.

11. The ultrasound imaging device of claim 10, wherein the micro controller unit is further configured for comparing each of the three real-time rotational components of the real-time rotational acceleration with the first preset threshold when a difference between any one of the three real-time linear components of the real-time linear acceleration and the corresponding initial linear component exceeds a second preset threshold; and
the micro controller unit is further configured for sending the probe activating instruction to the ultrasound host when any one of the three real-time rotational components of the real-time rotational acceleration exceeds the first preset threshold.

12. The ultrasound imaging device of claim 9, wherein the micro controller unit is further configured for updating an original displacement count value to obtain an updated displacement count value when the real-time acceleration meets the preset condition, and sending the updated displacement count value to the ultrasound host; and
the ultrasound host is further configured for activating the ultrasound probe according to the updated displacement count value.

13. The ultrasound imaging device of claim 12, wherein the micro controller unit is further configured for increasing the original displacement count value by a preset value to obtain the updated displacement count value.

14. The ultrasound imaging device of claim 9, wherein the motion sensor is further configured for sending an interrupt instruction to the micro controller unit when the motion sensor perceives a gravity acceleration of the ultrasound probe before acquiring the real-time acceleration of the ultrasound probe detected by the motion sensor; and
the micro controller unit is further configured for reading an initial acceleration of the ultrasound probe from a register of the motion sensor in response to a received interrupt instruction.

15. The ultrasound imaging device of claim 9, wherein the micro controller unit is further configured for reading a register of the motion sensor in real time to obtain the real-time acceleration of the ultrasound probe.

16. A micro controller unit, comprising a memory and a processor, wherein a computer program is stored in the memory, and the processor is configured to execute the computer program to perform the method of claim 1.

17. The micro controller unit of claim 16, wherein the real-time linear acceleration comprises three real-time linear components and the real-time rotational acceleration comprises three real-time rotational components; the preset condition further comprises that any one of the three real-time linear components of the real-time linear acceleration is not matched with a corresponding initial linear component, and any one of the three real-time rotational components of the real-time rotational acceleration exceeds the first preset threshold.

18. The micro controller unit of claim 17, wherein in the case that the real-time acceleration meets the preset condition, sending the probe activating instruction to the ultrasound host further comprises:
when a difference between any one of the three real-time linear components of the real-time linear acceleration and the corresponding initial linear component exceeds a second preset threshold, comparing each of the three real-time rotational components of the real-time rotational acceleration with the first preset threshold; and
when any one of the three real-time rotational components of the real-time rotational acceleration exceeds the first preset threshold, sending the probe activating instruction to the ultrasound host.

19. The micro controller unit of claim 16, wherein in the case that the real-time acceleration meets the preset condition, sending the probe activating instruction to the ultrasound host further comprises:
in the case that the real-time acceleration meets the preset condition, updating an original displacement count value to obtain an updated displacement count value; and
sending the updated displacement count value to the ultrasound host, and activating, by the ultrasound host, the ultrasound probe according to the updated displacement count value.

20. A computer-readable storage medium, storing a computer program, wherein the computer program is executed by a processor to perform the method of claim 1.
